(19) United States
(12) Patent Application Publication
(10) Pub. No.: US 2018/0271768 A1
(43) Pub. Date: Sep. 27, 2018

(54) BIOCELLULOSE SLURRY, AND DRY BIOCELLULOSE MATERIAL, METHOD FOR MANUFACTURING DRY BIOCELLULOSE MATERIAL AND SKIN CARE FILM USING THE SAME

(71) Applicant: TCI CO., LTD, Taipei (TW)

(72) Inventors: Yung-Hsiang LIN, Taipei (TW); Wei-Wen KUO, Taipei (TW)

(21) Appl. No.: 15/926,610

(22) Filed: Mar. 20, 2018

Related U.S. Application Data
(60) Provisional application No. 62/474,476, filed on Mar. 21, 2017.

(51) Int. Cl. A61K 8/73; A61K 8/02; A61Q 19/00

(52) U.S. Cl. CPC A61K 8/731; A61Q 19/00; A61K 8/0212

(57) ABSTRACT

A bio-cellulose slurry is provided. The bio-cellulose slurry comprises the following components: bio-cellulose; a backbone fiber selected from the group consisting of rayon fibers, polyethylene terephthalate (PET) fibers, nylon fibers and combinations thereof; an adhesive fiber selected from polyethylene composite fibers; an adhesion promoter selected from the group consisting of polyamides, low density polyethylenes and combinations thereof; a humectant selected from polyols; and a dispersion medium. The bio-cellulose slurry is useful in manufacturing a dry bio-cellulose material with outstanding water absorption rate and wet strength.

### CROSS REFERENCE TO RELATED APPLICATIONS

This application claims the benefit of U.S. Provisional Application No. 62/474,476 filed on Mar. 21, 2017, the subject matters of which are incorporated herein in their entirety by reference.

### BACKGROUND OF THE INVENTION

### Field of the Invention

The present invention provides a bio-cellulose slurry, which is useful in manufacturing a dry bio-cellulose material having an outstanding water absorption rate and wet strength. Therefore, the present invention also provides a method for manufacturing a dry bio-cellulose material, a dry bio-cellulose material, and a skin care film using the dry bio-cellulose material.

### Descriptions of the Related Art

Bio-cellulose is a natural fiber synthesized through the fermentation of microbes. The diameter of the bio-cellulose is only 20 nm-1000 times thinner than human hair. The bio-cellulose forms a bio-cellulose membrane after continuous fermentation. Since the bio-cellulose membrane has an extremely smooth surface, the texture of skin, and an outstanding water retention capacity and elasticity, it is particularly useful in cosmetic and biomedical fields. For example, the bio-cellulose membrane can be used as a substrate of a facial mask for carrying skin care ingredients.

However, the bio-cellulose membrane must be kept wet, and unlike a non-woven fabric facial mask substrate, cannot be manufactured into a dry cloth roll, because once the bio-cellulose membrane dried, it loses water absorption ability. Due to this disadvantage, the bio-cellulose membrane cannot be manufactured in a continuous manner and is inconvenient in storage and transportation. Furthermore, after the bio-cellulose membrane being cut into desired shape, the unwanted material cut from the bio-cellulose membrane are not properly used but directly thrown away. This is really wasteful and causes additional problems in handling the unwanted material.

### SUMMARY OF THE INVENTION

In view of the aforementioned technical problems, the present invention uses such unwanted bio-cellulose material to provide a bio-cellulose slurry, which is useful in manufacturing a new dry bio-cellulose material that can be continuously manufactured in the form of a roll product and therefore is convenient in storage and transportation. Moreover, the dry bio-cellulose material has an outstanding water absorption rate and wet strength and therefore is a particularly suitable substrate to a skin care film.

Therefore, an objective of the present invention is to provide a bio-cellulose slurry. The bio-cellulose slurry comprises:

bio-cellulose;

a backbone fiber selected from the group consisting of rayon fibers, polyethylene terephthalate (PET) fibers, nylon fibers and combinations thereof;

an adhesive fiber selected from polyethylene composite fibers;

an adhesion promoter selected from the group consisting of polyamides, low density polyethylenes and combinations thereof;

a humectant selected from polyols; and

a dispersion medium.

In some embodiments of the bio-cellulose slurry of the present invention, the backbone fiber is a combination of a rayon fiber and a polyethylene terephthalate fiber, the adhesive fiber is a polyethylene-polyester composite fiber, the adhesion promoter is a polyamide, and the humectant is glycerol.

In some embodiments of the bio-cellulose slurry of the present invention, the amount of the backbone fiber is about 4 parts by weight to about 12 parts by weight per 100 parts by weight of the bio-cellulose, the amount of the adhesive fiber is about 2 parts by weight to about 8 parts by weight per 100 parts by weight of the bio-cellulose, and the amount of the adhesion promoter is about 4 parts by weight to about 10 parts by weight per 100 parts by weight of the bio-cellulose. Preferably, the amount of the backbone fiber is about 6 parts by weight to about 8 parts by weight per 100 parts by weight of the bio-cellulose, the amount of the adhesive fiber is about 3 parts by weight to about 5 parts by weight per 100 parts by weight of the bio-cellulose, and the amount of the adhesion promoter is about 6 parts by weight to about 8 parts by weight per 100 parts by weight of the bio-cellulose.

In some embodiments of the bio-cellulose slurry of the present invention, the volume ratio of the dispersion medium to the humectant is about 10:1 to about 20:1 and more specifically about 10:1 to about 15:1.

Another objective of the present invention is to provide a method for manufacturing a dry bio-cellulose material. The method comprises the following steps in sequence:

a film-making step, depositing the aforementioned bio-cellulose slurry on a porous supporting substrate to provide a bio-cellulose material film on the porous supporting substrate;

a drying step, removing at least a portion of the dispersion medium from the bio-cellulose material film; and a hot-pressing step, hot-pressing the bio-cellulose material film to provide the dry bio-cellulose material.

In some embodiments of the method of the present invention, the drying step is performed at a temperature from about 80° C. to about 100° C. for about 1 min to about 10 min.

In some embodiments of the method of the present invention, the hot-pressing step is performed at a temperature from about 120° C. to about 150° C. for about 5 sec to about 30 sec, wherein the surface of the bio-cellulose material film which is not in contact with the porous supporting substrate is preferably hot-pressed for about 10 sec to about 20 sec, and the surface of the bio-cellulose material film which is in contact with the porous supporting substrate is preferably hot-pressed for about 5 sec to about 10 sec.

Yet another objective of the present invention is to provide a dry bio-cellulose material. The dry bio-cellulose material is manufactured by using the aforementioned method and has a water absorption rate of at least about 800% and a wet strength of at least about 200 g/75 cm².

Still another objective of the present invention is to provide a skin care film. The skin care film comprises a substrate and a skin care ingredient carried on the substrate, wherein the substrate is the aforementioned dry bio-cellulose material.

To render the above objectives, the technical features and advantages of the present invention more apparent, the present invention will be described in detail with reference to some embodiments hereinafter.

### BRIEF DESCRIPTION OF THE DRAWINGS

FIG. 1 is a schematic diagram showing a film-making equipment suitable for performing the method for manufacturing a dry bio-cellulose material according to the present invention.

### DESCRIPTION OF THE PREFERRED EMBODIMENT

Hereinafter, some embodiments of the present invention will be described in detail. However, without departing from the spirit of the present invention, the present invention may be embodied in various embodiments and should not be limited to the embodiments described in the specification.

Unless it is additionally explained, the expressions “a,” “the,” or the like recited in the specification (especially in the claims) should include both the singular and the plural forms.

As used herein, the term “about” in quantitative terms refers to plus or minus an amount that is general and reasonable to persons skilled in the art.

In the appended drawings, for clarity, the thickness of each layer and each area may be exaggerated.

As indicated in the above, the development of bio-cellulose membrane faces several problems, including the difficulty in continuous manufacture, storage and transportation due to the necessity of keeping the bio-cellulose membrane wet, and the problem in handling the unwanted material. The present invention provides a solution for the aforementioned technical problems. Specifically, the present invention provides a bio-cellulose slurry, which can be used to manufacture a new dry bio-cellulose material through general paper-making technique. The manufactured dry bio-cellulose material not only has the advantages of bio-cellulose, but also has an outstanding water absorption rate and wet strength. Therefore, the dry bio-cellulose material is a particularly suitable substrate to a skin care film, like a facial mask.

### 1. BIO-CELLULOSE SLURRY

The bio-cellulose slurry of the present invention comprises bio-cellulose, a backbone fiber, an adhesive fiber, an adhesion promoter, a humectant, and a dispersion medium as essential components. The bio-cellulose slurry can be used to manufacture a dry bio-cellulose material through general paper-making technique.

The backbone fiber improves the structural strength and material properties of the manufactured bio-cellulose material. The backbone fiber is selected from the group consisting of rayon fibers, polyethylene terephthalate (PET) fibers, nylon fibers and combinations thereof. The adhesive fiber is selected from polyethylene composite fibers and gives bonding effect to fibers. Examples of the adhesive fiber include but are not limited to polyethylene-polyester core-sheath composite fibers and polyethylene-polyester side by side composite fibers. The adhesion promoter enhances the bonding strength between fibers and is selected from the group consisting of polyamides, low density polyethylenes and combinations thereof. The humectant improves the water absorption ability of the manufactured bio-cellulose material is selected from polyols. The dispersion medium can be any inert medium that can dissolve or disperse the components of the bio-cellulose slurry but does not react with the components. An example of the dispersion medium is water. In some embodiments of the present invention, the backbone fiber is a combination of a rayon fiber and a polyethylene terephthalate fiber, the adhesive fiber is a polyethylene-polyester core-sheath composite fiber (core: polyester; sheath: polyethylene), the adhesion promoter is a polyamide, the humectant is glycerol, and the dispersion medium is water.

In the bio-cellulose slurry of the present invention, the amount of the backbone fiber is preferably about 4 parts by weight to about 12 parts by weight, and more preferably about 6 parts by weight to about 8 parts by weight per 100 parts by weight of the bio-cellulose. The amount of the adhesive fiber is preferably about 2 parts by weight to about 8 parts by weight, and more preferably about 3 parts by weight to about 5 parts by weight per 100 parts by weight of the bio-cellulose. The amount of the adhesion promoter is preferably about 4 parts by weight to about 10 parts by weight, and more preferably about 6 parts by weight to about 8 parts by weight per 100 parts by weight of the bio-cellulose. The volume ratio of the dispersion medium to the humectant is preferably about 10:1 to about 20:1, and more preferably about 10:1 to about 15:1. Under the aforementioned preferred ranges, the bio-cellulose material manufactured from the bio-cellulose slurry of the present invention is provided with a better water absorption rate and wet strength.

In the bio-cellulose slurry of the present invention, the length of the bio-cellulose, backbone fiber or adhesive fiber may be adjusted by persons having ordinary skill in the art depending on the need. However, the backbone fiber preferably has a length of at least about 2 mm like about 3 mm to about 30 mm, such as 4 mm, 5 mm, 6 mm, 7 mm, 8 mm, 9 mm, 10 mm, 12 mm, 15 mm, 20 mm or 25 mm, in terms of the structural strength of the manufactured dry bio-cellulose material, but the present invention is not limited thereto. The adhesive fiber preferably has a length of at least about 1 mm like about 2 mm to about 25 mm, such as 3 mm, 4 mm, 5 mm, 6 mm, 7 mm, 8 mm, 9 mm, 10 mm, 12 mm, 15 mm or 20 mm, in terms of the bonding strength between fibers, but the present invention is not limited thereto.

The bio-cellulose slurry of the present invention may be prepared by mixing and dispersing the essential components of the bio-cellulose slurry, including the bio-cellulose, backbone fiber, adhesive fiber, adhesion promoter and humectant, and other optional additives in the dispersion medium. The additives can be any additive that is usually added into a skin care film. The ratio of the dispersion medium to the rest components, including the bio-cellulose, backbone fiber, adhesive fiber and adhesion promoter, is not particularly limited and can be adjusted by persons having ordinary skill in the art depending on the need. For example, the bio-cellulose slurry of the present invention may be prepared into a concentrated form by dispersing the components of the bio-cellulose slurry in a small amount of the dispersion medium, such that the bio-cellulose slurry can be transported and sold as a concentrated slurry and diluted by using the dispersion medium right before use.

### 2. METHOD FOR MANUFACTURING BIO-CELLULOSE MATERIAL

The present invention also provides a method for manufacturing a dry bio-cellulose material from the aforementioned bio-cellulose slurry. The method comprises a film-making step, a drying step, and a hot-pressing step in the given order. In the film-making step, the bio-cellulose slurry is deposited on a porous supporting substrate to provide a bio-cellulose material film on the porous supporting substrate. In the drying step, at least a portion of the dispersion medium is removed from the bio-cellulose material film. In the hot-pressing step, the bio-cellulose material film is hot-pressed to bind the fibers together to obtain the dry bio-cellulose material.

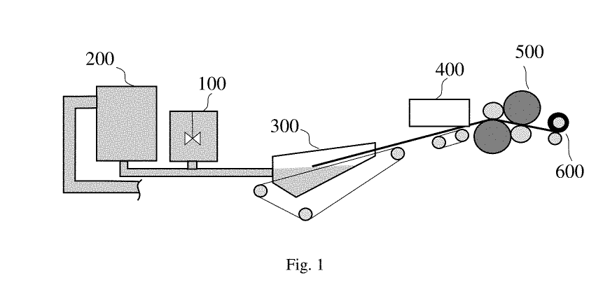

The method of the present invention can be performed via general paper-making technique. For example, the film-making equipment shown in FIG. 1 may be used to perform the method for manufacturing a dry bio-cellulose material according to the present invention. In detail, the bio-cellulose minced through a mincer in advance, backbone fiber, adhesive fiber, adhesion promoter and humectant are uniformly mixed in a mixing tank 100. Then the resultant mixture is uniformly mixed with water from a circulating water tank 200 in a film-making tank 300 to provide a bio-cellulose slurry. Afterward, a film-making step is performed to deposit bio-cellulose slurry on a blanket (not shown) to form a bio-cellulose material film. The bio-cellulose material film is conveyed to an oven 400 via a water absorbing conveyer belt and dried in the oven 400 to remove a portion of the dispersion medium. After that, the dried bio-cellulose material film is hot-pressed by hot-pressing rollers 500 to obtain a dry bio-cellulose material. The obtained dry bio-cellulose material can be wound into roll material via a winding device 600 and stored.

In the method of the present invention, the drying step is preferably performed at a temperature from about 80° C. to about 100° C., such as 85° C., 90° C. or 95° C., for about 1 min to about 10 min, such as 2 min, 3 min, 4 min, 5 min, 6 min, 7 min, 8 min or 9 min. The hot-pressing temperature should be at least higher than the melting points of the adhesive fiber and adhesion promoter and is preferably from about 120° C. to about 150° C., such as 125° C., 130° C., 135° C., 140° C. or 145° C., in order to provide the desired bonding effect. The hot-pressing time is preferably about 5 sec to about 30 sec, such as 15 sec, 20 sec or 25 sec. Further, it is preferred to hot-pressing both surfaces of the bio-cellulose material film, wherein the surface of the bio-cellulose material film which is not in contact with the porous supporting substrate is preferably hot-pressed for about 10 sec to about 20 sec, and the surface of the bio-cellulose material film which is in contact with the porous supporting substrate is preferably hot-pressed for about 5 sec to about 10 sec. Under the aforementioned drying and hot-pressing conditions, the dry bio-cellulose material manufactured by the method of the present invention is provided with a better water absorption rate and wet strength.

The method of the present invention is advantageous in manufacturing cost, storage and transportation because it can be performed in a continuous manner and the manufactured dry bio-cellulose material can be stored in roll form.

3. Dry Bio-Cellulose Material

The present invention also provides a dry bio-cellulose material manufactured by the aforementioned method. The dry bio-cellulose material can be continuously manufactured and wound into roll material and therefore solves the storage and transportation problems of conventional bio-cellulose material due to the necessity of keeping the bio-cellulose membrane wet. In the preferred embodiments of the present invention, the dry bio-cellulose material has a water absorption rate of at least about 800% and a wet strength of at least about 200 g/75 cm², such as a water absorption rate of 850%, 950%, 1000%, 1050%, 1100%, 1150%, 1200%, 1250%, 1300%, 1350%, 1400%, 1450% or 1500%, and a wet strength of 250 g/75 cm², 300 g/75 cm², 350 g/75 cm², 400 g/75 cm², 450 g/75 cm², 500 g/75 cm², 550 g/75 cm², or 600 g/75 cm². The water absorption rate and wet strength here are measured according to the methods provided in the following Examples.

### 4. SKIN CARE FILM

The dry bio-cellulose material of the present invention has an outstanding water absorption rate and wet strength and therefore can be used as a substrate of a skin care film. Therefore, the present invention also provides a skin care film comprising a substrate and a skin care ingredient carried on the substrate, wherein the aforementioned dry bio-cellulose material is used as the substrate of the skin care film. Examples of the skin care film include but are not limited to a facial mask, a finger mask and a foot mask. The skin care ingredient of the skin care film is not particularly limited and can be any ingredient known in the art. Examples of the skin care ingredient include tea polyphenols and hyaluronic acid, but the present invention is not limited thereto. The skin care film of the present invention has the advantages of general bio-cellulose, and moreover, it is capable of containing a lot of skin care ingredient while maintaining an outstanding strength due to the outstanding water sorption rate and wet strength of the substrate.

### 5. EXAMPLES

5.1. Testing Methods

The present invention will be further illustrated by the embodiments hereinafter, wherein the measuring instruments and methods are respectively as follows:

[Water Absorption Rate Test]

A 5 cm×5 cm sample is placed in an oven and dried at 60° C. for 24 hr. After that, the weight of the sample is measured to obtain the dry weight. Next, the sample is dipped in flowing water for 5 min and taken out and hung for 1 min. Then the weight of the dipped sample is measured to obtain the wet weight. The water absorption rate of the sample is calculated according to the following equation:

Water absorption rate (%)=(wet weight/dry weight)×100%

[Wet Strength Test]

The water strength test is performed by using a universal strength tester (chuck velocity: 200 cm/min), wherein a 5 cmx15 cm sample is used and the sample absorbs water in an amount equal to its own weight before being subjected to the water strength test.

5.2. Manufacture of Dry Bio-Cellulose Material

A dry bio-cellulose material was manufactured by using the film-making equipment shown in FIG. 1. In detail, the bio-cellulose minced through a mincer in advance, backbone fiber, adhesive fiber, adhesion promoter and humectant are uniformly mixed in the mixing tank 100 according to the ratio shown in Table 1. Then the resultant mixture was uniformly mixed with water from the circulating water tank 200 in the film-making tank 300 to provide a bio-cellulose slurry. Afterward, a film-making step was performed to deposit bio-cellulose slurry on a PET blanket (not shown) to form a bio-cellulose material film. The bio-cellulose material film was conveyed to the oven 400 via a water absorbing conveyer belt and dried in the oven 400 (drying temperature: 80° C.; drying time: 3 min). After that, the dried bio-cellulose material film was hot-pressed by the hot-pressing rollers 500 to obtain a dry bio-cellulose material (hot-pressing temperature: 120° C.; hot-pressing time: the front surface was hot-pressed for 10 sec and the rear surface was hot-pressed for 5 sec, wherein the front surface is the surface of the bio-cellulose material film which is not in contact with the porous supporting substrate, and the rear surface is the surface of the bio-cellulose material film which is in contact with the porous supporting substrate). The obtained dry bio-cellulose material was wound into roll material via the winding device 600 and stored.

TABLE 1
Constitution of bio-cellulose slurry

| | Components | Amounts | |
|---|---|---|---|
| Bio-cellulose | | 32 | g |
| Backbone | PET fiber (length: 10 mm; Model No.: t200, | 1 | g |
| fiber | available from Far Eastern New Century | | |
| | Company) | | |
| | Rayon fiber (length: 8 mm; Model No.: short | 1 | g |
| | cut, available from Formosa Chemical & Fibre | | |
| | Company) | | |
| Adhesive | ES FIBERVISIONS ® ETC (ES-Tendon-C) | 1.5 | g |
| fiber | fiber-containing polyethylene-polyester | | |
| | core-sheath composite fibers (length: 5 mm; | | |
| | Model No.: A313, available from ES | | |
| | FIBERVISIONS Company; core: polyester; | | |
| | sheath: polyethylene) | | |
| Adhesion | Polyamide powder (Model No.: PA 2200, | 2 | g |
| promoter | available from EOS Company) | | |
| Humectant | Glycerol | 100 | mL |
| Dispersion | Water | 1000 | mL |
| medium | | | |

5.3. Property Tests of Dry Bio-Cellulose Material

The basis weight, thickness, water absorption rate and wet strength of the manufactured dry bio-cellulose material were measured, and the results are tabulated in the following Table 2.

TABLE 2
Properties of dry bio-cellulose material

| Basis weight | Thickness | Water absorption | Wet strength |
|---|---|---|---|
| (gsm) | (mm) | rate (%) | (g/75 cm²) |
| 100 | 3.02 | 1380 | 480 |

As shown in Table 2, the dry bio-cellulose material of the present invention has outstanding water absorption rate (1380%) and wet strength (480 g/75 cm²) and therefore is a suitable substrate for a skin care film.

The above examples are used to illustrate the principle and efficacy of the present invention and show the inventive features thereof. People skilled in this field may proceed with a variety of modifications and replacements based on the disclosures and suggestions of the invention as described without departing from the principle and spirit thereof. Therefore, the scope of protection of the present invention is that as defined in the claims as appended.

### BRIEF DESCRIPTION OF NUMERAL REFERENCES

100: mixing tank
200: circulating water tank
300: film-making tank
400: oven
500: hot-pressing roller
600: winding device

What is claimed is:

1. A bio-cellulose slurry, comprising:
bio-cellulose;
a backbone fiber selected from the group consisting of rayon fibers, polyethylene terephthalate (PET) fibers, nylon fibers and combinations thereof;
an adhesive fiber selected from polyethylene composite fibers;
an adhesion promoter selected from the group consisting of polyamides, low density polyethylenes and combinations thereof;
a humectant selected from polyols; and
a dispersion medium.

2. The bio-cellulose slurry of claim 1, wherein the backbone fiber is a combination of a rayon fiber and a polyethylene terephthalate fiber, the adhesive fiber is a polyethylene-polyester composite fiber, the adhesion promoter is a polyamide, and the humectant is glycerol.

3. The bio-cellulose slurry of claim 1, wherein the amount of the backbone fiber is about 4 parts by weight to about 12 parts by weight per 100 parts by weight of the bio-cellulose, the amount of the adhesive fiber is about 2 parts by weight to about 8 parts by weight per 100 parts by weight of the bio-cellulose, and the amount of the adhesion promoter is about 4 parts by weight to about 10 parts by weight per 100 parts by weight of the bio-cellulose.

4. The bio-cellulose slurry of claim 3, wherein the amount of the backbone fiber is about 6 parts by weight to about 8 parts by weight per 100 parts by weight of the bio-cellulose, the amount of the adhesive fiber is about 3 parts by weight to about 5 parts by weight per 100 parts by weight of the bio-cellulose, and the amount of the adhesion promoter is about 6 parts by weight to about 8 parts by weight per 100 parts by weight of the bio-cellulose.

5. The bio-cellulose slurry of claim 1, wherein the volume ratio of the dispersion medium to the humectant is about 10:1 to about 20:1.

6. The bio-cellulose slurry of claim 5, wherein the volume ratio of the dispersion medium to the humectant is about 10:1 to about 15:1.

7. A method for manufacturing a dry bio-cellulose material, comprising the following steps in sequence:
a film-making step, depositing the bio-cellulose slurry of claim 1 on a porous supporting substrate to provide a bio-cellulose material film on the porous supporting substrate;
a drying step, removing at least a portion of the dispersion medium from the bio-cellulose material film; and
a hot-pressing step, hot-pressing the bio-cellulose material film to provide the dry bio-cellulose material.

8. The method of claim 7, wherein the drying step is performed at a temperature from about 80° C. to about 100° C. for about 1 min to about 10 min.

9. The method of claim 7, wherein the hot-pressing step is performed at a temperature from about 120° C. to about 150° C. for about 5 sec to about 30 sec.

10. The method of claim 9, wherein, in the hot-pressing step, the surface of the bio-cellulose material film which is not in contact with the porous supporting substrate is hot-pressed for about 10 sec to about 20 sec, and the surface of the bio-cellulose material film which is in contact with the porous supporting substrate is hot-pressed for about 5 sec to about 10 sec.

11. A dry bio-cellulose material, which is manufactured by using the method of claim 7.

12. The dry bio-cellulose material of claim 11, which has a water absorption rate of at least about 800% and a wet strength of at least about 200 g/75 cm².

13. A dry bio-cellulose material, which is manufactured by using the method of claim 8.

14. The dry bio-cellulose material of claim 13, which has a water absorption rate of at least about 800% and a wet strength of at least about 200 g/75 cm².

15. A dry bio-cellulose material, which is manufactured by using the method of claim 9.

16. The dry bio-cellulose material of claim 15, which has a water absorption rate of at least about 800% and a wet strength of at least about 200 g/75 cm².

17. A dry bio-cellulose material, which is manufactured by using the method of claim 10.

18. The dry bio-cellulose material of claim 17, which has a water absorption rate of at least about 800% and a wet strength of at least about 200 g/75 cm².

19. A skin care film, comprising a substrate and a skin care ingredient carried on the substrate, wherein the substrate is the dry bio-cellulose material of claim 11.

20. A skin care film, comprising a substrate and a skin care ingredient carried on the substrate, wherein the substrate is the dry bio-cellulose material of claim 12.
